(19) United States
(12) Patent Application Publication
(10) Pub. No.: US 2006/0173718 A1
(43) Pub. Date: Aug. 3, 2006

(54) Medical information card and system and method for using same

(76) Inventor: Daniel J. Murphy, Sterling, MA (US)

Correspondence Address: Mirick, O'Cormell,;Demallie & Lounges. LLP, 100 Front, Worcester, MA, 01608, US

(21) Appl. No.: 11/346,133

(22) Filed: Feb. 2, 2006

Related U.S. Application Data
(60) Provisional application No. 60/649,349, filed on Feb. 2, 2005.

(51) Int. Cl. G06Q 10/00; A61B 5/00

(52) U.S. Cl. 705/2; 600/300

(57) ABSTRACT

A system for ensuring that a person receives individual-specific health care and avoiding adverse drug reactions, generally comprises: an information card on which critical need medical information, specific to said person, is be stored, and a database that can be electronically accessed by the person so that the person can update their own information stored on the card.

### CROSS-REFERENCE

This is a continuation-in-part of U.S. Application Ser. No. 60/649,349 filed on Feb. 2, 2005.

### FIELD OF THE INVENTION

This invention relates to medical information cards and more specifically to a system and methods for ensuring that a person receives individual-specific health care by using such cards to communicate the individual's health care information for them.

### BACKGROUND OF THE INVENTION

In the medical industry today there are many treatment options available for patients, especially through prescription drugs. With the variety of drugs that are available, it is also becoming difficult to administer treatment that would avoid adverse drug reactions (ADRs). ADRs are a variety of issues that are associated with drugs administered for treatment. These issues include allergies to drugs and drug-to-drug interactions, where a drug that is administered has a negative reaction with a drug that is already in the patient's system. There are millions of ADR injuries and hundreds of thousands of deaths each year due to complications of ADRs. Most of these ADR situations can be prevented with better communication.

At the present time, there is not any way for emergency personnel to know of an emergency patient's medical history or prescriptions if the patient is unconscious or otherwise unable to communicate with the emergency personnel.

There is also not any way for patients to manage information about their own medical history and prescriptions in a manner that is accurate, efficient, or current, and certainly not in any manner that enables the patients to carry such information with them at all times in a purse, wallet or pocket.

### SUMMARY OF THE INVENTION

It is therefore a primary object of this invention to provide system for ensuring that a person receives individual-specific health care and avoiding adverse drug reactions.

It is a further object of this invention to provide a system that enables a person to communicate efficiently and effectively with a network of health services personnel who have access to the person's medical information, wherein the level of access to the person's information is controlled by the person.

The system and methods of the invention were designed to address many of the problems associated with health care systems in which health care providers are unable to provide adequate emergency services to a given patient because the providers do not have readily accessible information specific to that patient.

A preferred embodiment of the system for ensuring that a person receives individual-specific health care and avoiding adverse drug reactions, generally comprises: an information card on which critical need medical information, specific to said person, may be stored; a means for storing said critical need medical information specific to said person on said card; a means for reading said critical need medical information for the purpose of providing a health-related service to said person in view of said information read from said card; and a means for updating said information stored on said card. The means for updating preferably comprises a database, of at least some of said critical need medical information, that is electronically accessible by said person, and a card printer, wherein said database may be accessible by said person via a computer networked system. The system may still further comprise a means for electronically reading said critical need medical information stored on said card.

The means for updating may also be a database, comprising information about a plurality of people, that is accessible by said person by entering a personal identification code specific to said person, and wherein said person can only access said critical need medical information of said person, wherein said means for updating allows said person to change said person's personal identification code.

The information card of the system may comprise an electronically-readable means for storing said medical information, wherein said electronically-readable means may comprise a magnetic strip, computer chip or a memory means selected from a group consisting of a memory card and memory stick. The means for updating may also comprises a telephonically accessible database whereby the person may update the information over the telephone and then a new card is mailed to the person with the updated information stored on the card.

The means for updating may also be a stand-alone personal computer and a computer program residing in said computer that enable said person to update said person's medical information by entering updated information into said computer to update said information on said card, wherein the system may comprise a means for creating a new card with said updated information stored on said new card.

The critical need medical information stored on the card preferably comprises at least one type of information specific to said person that is selected from a group consisting of: allergies, prescriptions used by said person, non-prescription medications used by said person, devices implanted in said person, ailments of said person, and said person's latest electrocardiogram. The card may still further comprise identification information specific to said person, wherein said identification information comprises at least one type of information specific to said person and selected from a group consisting of: said person's primary care physician, personal emergency contact information, organ donation information, personal residential information, contact information for closest relative, health insurance information, and date critical need medical information was last updated.

Another preferred embodiment of the system of the invention, for ensuring that a person receives individual-specific health care and avoiding adverse drug reactions, comprises: an information card on which critical need medical information, specific to said person, may be stored; a means for storing said critical need medical information specific to said person on said card; and a means for enabling said person to electronically update said person's own medical information stored on said card, wherein said means for storing may comprise an imprinting device for imprinting a plurality of raised letters and numbers on a surface of said card corresponding to at least some of said critical need medical information. The means for storing may also comprise a thermal printing device for printing some or all of the individual's information on a surface of the card.

Similarly, the means for enabling said person to electronically update said person's own medical information stored on said card preferably comprises a database electronically accessible by said person via a computer networked system, wherein the system may further comprise a means for creating a new information card on which said person's updated medical information is stored.

The system may also comprise a means for said person to communicate, via a computer networked system, with one or more value-added service structures capable of assisting said person to create, change and maintain said person's information, wherein said service structures comprises one or more entities selected from a group consisting of: call-centers, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities, and caregivers.

The system may also comprise a means for managing information exchange between said person and one or more service entities selected from a group consisting of: call-centers, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities, and caregivers.

The means for enabling said person to electronically update said person's own medical information stored on said card may comprise one or more kiosk stations, further comprising an electronic database accessible via a computer networked system and wherein said kiosk stations comprise a means for accessing said database via said computer networked system.

Still further, the system may comprise a means for communicating at least a portion of said critical need medical information of said person to one or more third parties designated by said person, wherein one or more of said designated third parties is selected from a group consisting of: family members, caregivers, doctor, dentists, nurses, medical specialists, hospital administrative personnel, healthcare facilities, managed healthcare companies, pharmacists, pharmacies, healthcare insurers, healthcare networks, nursing homes, assisted living facilities, home healthcare organizations, call center or other value-add organizations. The means for communicating is selected from a group consisting of: wired computer networked system, wireless computer networked system, satellite, cable, telephone, facsimile, and mail.

### BRIEF DESCRIPTION OF THE DRAWINGS

Other objects, features and advantages will occur to those skilled in the art from the following description of the preferred embodiments and the accompanying drawings in which:

FIG. 1 is a top view of a preferred embodiment of the information card used in the system of the invention;

FIG. 2 is a schematic diagram of a preferred embodiment of the system of the invention;

FIG. 3 is a schematic diagram of another preferred embodiment of the system of the invention; and

FIG. 4 is a schematic diagram of yet another preferred embodiment of the system of the invention.

### DETAILED DESCRIPTION OF THE PREFERRED EMBODIMENTS AND METHODS

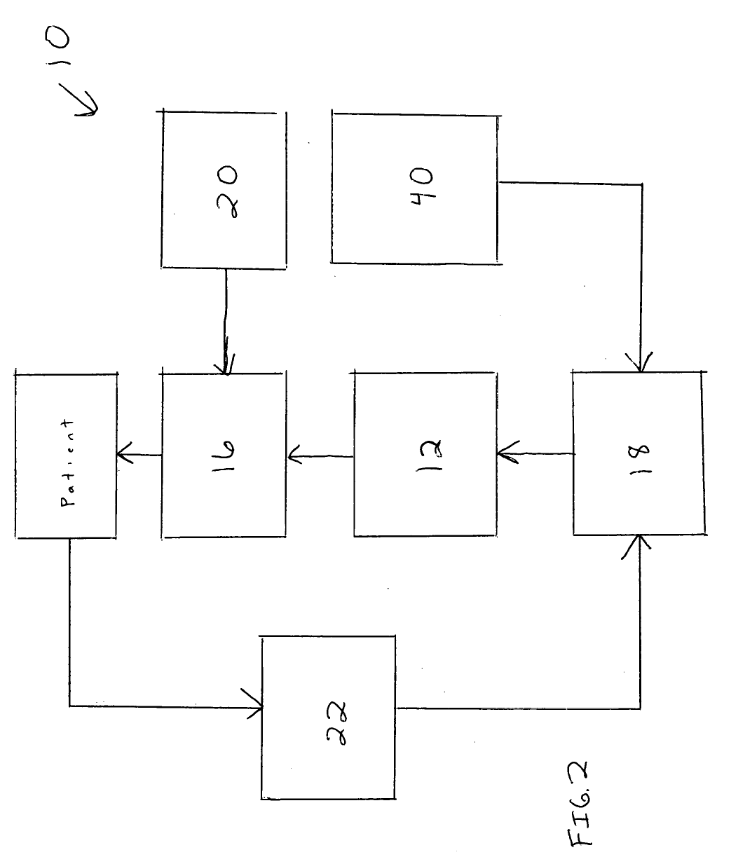

The invention features a system for ensuring that a person receives individual-specific health care and avoids adverse drug reactions. The system, shown and generally referred to in FIG. 2 at system 10, features an information card 16 on which critical need medical information, specific to said person, is stored. The system utilizes a means for storing 12 the critical need medical information specific to said person on said card. The means for storing may be an imprinting device for imprinting raised letters and numbers on the surface of the card or an electronic device for storing data on a magnetic strip provided on the card, or in a computer chip or memory card or stick that is inserted or embedded in the card.

In the latter instance, the card would need to comprise more than a plastic card (typically associated with credit cards and other similar cards). For example, the card may comprise a very thin personal computing device capable of reading and digitally displaying information digitally saved on a chip or memory device in the personal computing device.

The means for reading 20 the critical need medical information may simply comprise the raised letters and numbers imprinted on a plastic card. The means for reading may additionally or alternatively comprise a magnetic strip reader if a magnetic storage strip is provided on the card, or a computer or personal computing device if a chip or memory device such as a memory card or memory stick is used to store the person's information.

The means for updating the information stored on the card is preferably computer 22. Computer 22 may be a computer connected to a computer-networked system such as a local area network, a wireless network, or the Internet. Computer 22 may also be a handheld personal computing device. Computer 22 may also be a stand-alone computer in which a software program resides that enables the user to manage the user's own medical information and allows the user to print their own card. The card printer may be a paper or laminated card, or even an embossed card if the user has access to a card embosser or imprinter.

The means for updating may also comprise a voice-activated or touch pad telephone system that allows the user to access the user's personal information via a telephone and to update or change the information by entering the information with voice commands or by using the telephone keypads.

The means for updating preferably comprises a database 18, which resides in the memory of computer 22 or which is accessible via the computer 22 or telephone system, of at least some of said critical need medical information. If the system is adapted for use by many people, the database used in the system stores information for any of the people that have signed up for an account in the system and who desire to obtain a personal information card on which their own critical need medical information is stored and updated. The database is preferably accessible by each account holder by entering a personal identification code or PIN number, specific to said person, whereby the person can only access the critical need medical information of that person. The system should also enable the user to change the user's own personal identification code or PIN.

As noted, the information card of the system may comprise an electronically-readable means for storing said medical information, wherein said electronically-readable means may comprise a magnetic strip, computer chip or a memory means selected from a group consisting of a memory card and memory stick. The means for updating may also comprise a telephonically accessible database whereby the person may update the information over the telephone and then a new card is mailed to the person with the updated information stored on the card.

The information Card of the invention is adapted to communicate critical information between a patient (individual) and a healthcare provider. The information card is particularly suited to store the necessary information needed to avoid ADRs, specifically with regard to drug allergies and drug-to-drug interactions. The information card is preferably carried by an individual and provides readers of the card with critical healthcare information about that individual. The information card of the invention is particularly advantageous for use by emergency medical personnel who rarely have access to or knowledge about an emergency patient's medical history, prescriptions, or allergies.

The card contains a persons' prescription information and other critical information that is needed in a healthcare emergency, so that proper and safe treatment can be provided. An emergency would include emergency room, ambulance or surgery. The card is intended to be carried by the person, by any suitable means, so that the information can be communicated at the individual's discretion or can be found on the person in instances in which the individual cannot communicate with the medical personnel.

The card may also be used in non-emergency situations where access to and communication of such information is advantageous. Non-emergency situations would include; scheduled doctor visits, visit to health care specialists, dentists. One embodiment of the card is a credit card style with the information printed on it. The card is intended to be carried by a person, in their wallet or pocketbook, and is shown at the person's discretion. The card is setup with information supplied by the individual. The information on the card is updated as needed by the individual via web, mail, email, fax or phone. The individual's information is stored on a centralized database to be accessed for editing purposes by the individual or third parties or entities designated by the individual or otherwise permitted to maintain this information.

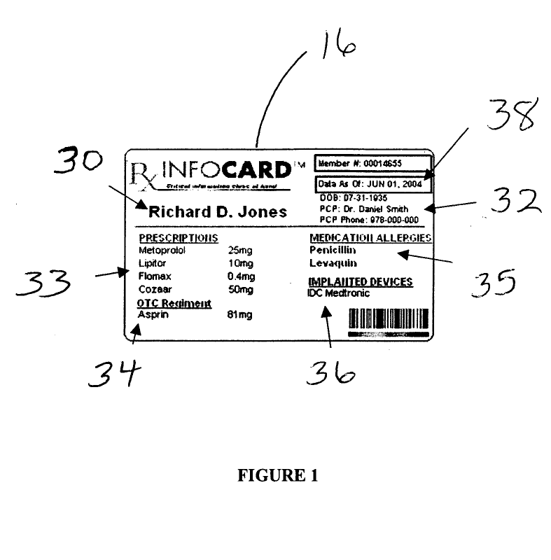

Although the specific dimensions of the card used in the system of the invention may be widely modified, depending on the application and use, one such embodiment of a plastic card type version, although not intended to be limiting in any way, is generally shown in FIG. 1. If a plastic credit card type version is used, the dimensions of such plastic cards may be approximately 3⅜″ wide×2⅛″ high and approximately 20 to 40 mils thick. The person's personal and medical information may be stored on the plastic card by thermally printing or embossing the information on the card. As noted, plastic cards may also have a magnetic strip on the card for storing and reading the data on the card.

The card may include an individual's information identifying the regimen of prescriptions, over the counter and holistic medication that the individual is taking regularly, along with any known allergies to medication and any implanted devices such as a pacemaker. Additionally, the card may list the individual's primary care physician and phone number and preferably indicates when information was last updated. As shown in FIG. 1, the card may have certain information printed on the face of the card, and/or information stored elsewhere on the card such as on a magnetic strip provided on the back of the card. With reference to FIG. 1, card 16 comprises line 30, which identifies the individual as the cardholder, line 32 which lists individual's primary doctor and contact information, line 33 which lists current prescription information of the individual, line 34 which identifies any over the counter medication the individual is taking regularly, line 35 which identifies medication that the individual is allergic to, line 36 which identifies any devices implanted in the individual, and line 38 which indicates when all of this information was last updated. The card may also include the individual's baseline electrocardiogram or a series of electrocardiograms, otherwise referred to as an EKGs or ECGs.

The information stored on the card may be source through a number of means and typically directly from the individual. If the database of patient information is remotely located from the individual, the information is sourced by the individual via phone, fax, mail, email, and online. The information is entered onto a centralized database where it is stored for future reference and updating. Once information is completed, if the system or database manager is an entity other than the individual, the updated information is processed to create a new updated card and card is mailed or otherwise sent to the individual.

If an individual's information changes, the individual updates their information directly on the database online or via phone, fax, mail or email. The information is accessed from the centralized database where edits are made and saved. Updated information is then sent to print an updated card if necessary and stored for future reference. Updated cards are again mailed or otherwise transmitted to individual.

The card should be carried by the individual and then presented by the individual, at their discretion, when a need for the information arises. Examples of such situations include but are not limited to: emergency situations such as ambulance and emergency room visits, or visits to doctors, specialists, dentists, pharmacists and pharmacies. The card may also be used by the individual to remind them of what prescriptions they are taking or should be taking and at what intervals. As an individual's information changes, they make the updates to the database and a new card is printed, if necessary.

The system of the invention provides a secure centralized prescription and medical information exchange system for communicating individuals' information to all healthcare environments. The system is established around a centralized database focusing on receiving, storing, editing, updating and retrieving information. An individual's information typically centers around critical need information that includes, but is not limited to, prescription information, drug allergies, over the counter regimens, holistic medication, implanted devices and baseline EKGs.

The information exchange system is utilized by individuals (general consumers), and with permission by an individual, may be accessed by caregivers, doctors, emergency rooms, ambulatory companies, dentists, nurses, medical specialists, hospital administrative personnel, healthcare facilities, managed healthcare companies, pharmacists, pharmacies, healthcare insurers, healthcare networks, nursing homes, home healthcare organizations and assisted living facilities. Use of the system includes, but is not limited to, identifying and confirming an individuals' drug regimen and baseline EKG to assist in all healthcare emergency and non-emergency situations.

As described, the information exchange system features a secure centralized database, such as database 18, that is preferably web enabled. Through this system, information is loaded into the database, either by the individual or by a system administrator. Initial information is entered through a registration process where the system gathers the information and creates an account. The system allows information to be retrieved through a login w/password format that identifies individual accounts for viewing, whether that is viewed by an individual, administrator, call center or any other authorized individual, facility or organization. Once logged on, all the information can be managed, updated, modified, deleted or more information can be added. This profile management is done through a set of edit screens and individual field edit options. New accounts that are set-up or any revisions to existing accounts are automatically sent to external service options. These options include; card printing, drug interaction screening, drug watch system, kiosk stations, managed information exchange and duplicate information exchange systems, notification to doctors, hospitals or other healthcare organizations or devices.

Additional features of the database include, but are not limited to, an import list of all possible drugs, enabling an automatic and accurate selection of known prescription drugs during the registration and profile management process to reduce errors.

As described, access to the database is web enabled and access to the database is given to an individual who sets-up the account. Additionally, with permission of the individual, access to that individual's personal information may be given to other value-added service structures 40 for assisting an individual to create, update and maintain their own information. Service structures include, but are not limited to, call-centers, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities and caregivers.

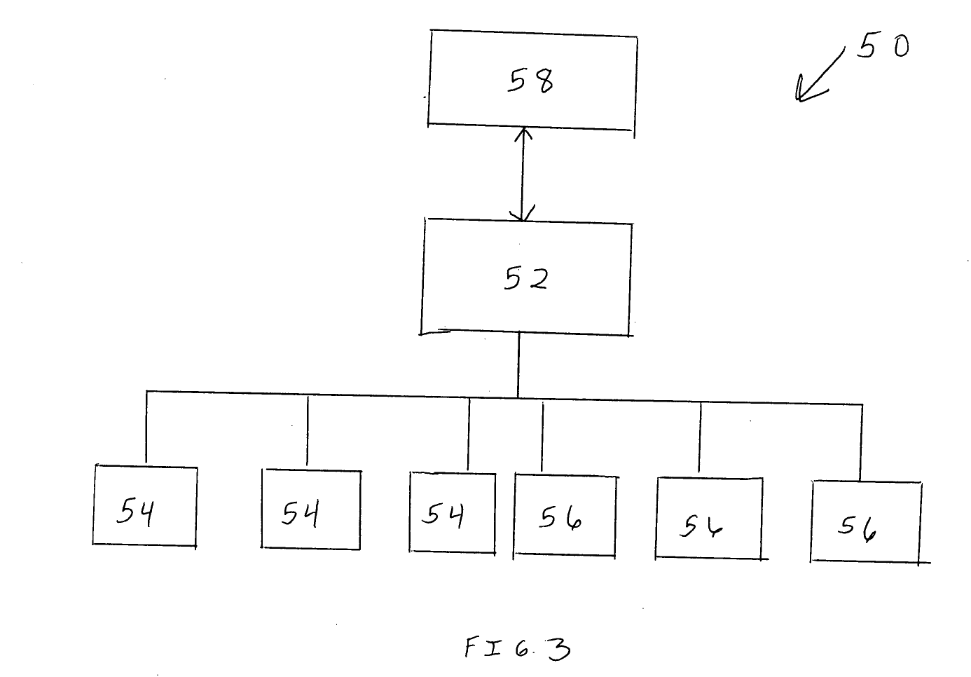

As shown in FIG. 3, system 50 may provide “campus privilege” which gives access to multiple accounts in the database 52. This allows organizations to have more administrative control to create, edit and manage more than one account that, in turn, allows an organization 58 to offer services to their patients or members 54 or other medical service providers 56 who provide services to members 54. Organizations include, but are not limited to, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities, home healthcare organizations, call centers or other value-add organizations.

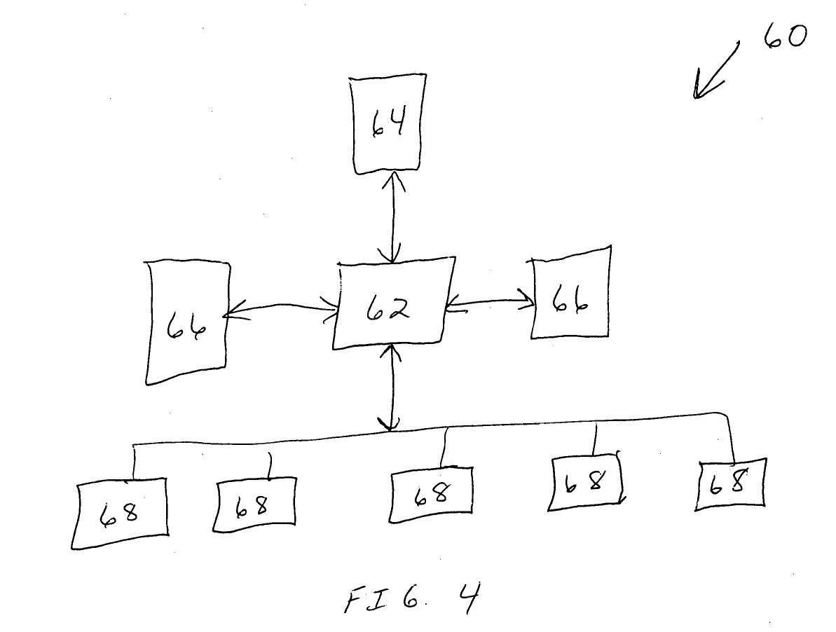

As shown in FIG. 4, system 60 of the invention may also provide “full access” which gives access to database 62 to all accounts 68 without administrative control. In addition to the account members 68, this access is used by healthcare organizations and services 66 where there is an emergency situation or emergency treatment is needed and they are in need of knowing an individuals' prescription regimen. Full access will determine if an individual's information is on the system. If an identified individual has an account on the system, then access to the account information is given either online, phone, fax or mail.

Kiosk stations may also be used as the means for updating as an independent or remote access station to access information on the database. The kiosks are connected to the database, giving an individual access to the database to create a new account or view existing account information for reference and updating. The kiosks also provide users with the ability to print out new and updated information cards and/or information sheets. Kiosk stations may be placed in any environment that makes it convenient for an individual to access their information on the database. Locations include; hospitals, pharmacies, department stores, senior centers, malls and any other convenient locations.

Additional services, such as drug interaction screening, may also be offered using the system of the invention. This screening cross checks the individual's regimen of medication for potential known drug-to-drug interactions. Upon discovery of a potential interaction, individuals are notified and encouraged to discuss findings with their primary care physician. Drug interactions indications may be sourced from the FDA, drug manufacturers, drug marketers and other drug information resources.

The system may also provide a drug watch system that cross checks an individual's medication regimen against any recalls or updated information on a specific drug. Once new information is discovered, notification is sent to the individual. The information is cross checked against information provided by the FDA, drug manufacturers, drug marketers and other drug information resources.

The system of the invention also provides a “managed information exchange” system that allows an individual to have their profile information communicated to a healthcare professional or organization. The communication of information may be in advance of any health services provided such as prior to their appointment. Use of this system may include, but is not limited to, regularly scheduled doctor visits, dentist visit, hospital stay, elective surgery, specialist visit, home healthcare visits or any other non-emergency or emergency situation. Communication of this information may be by email, phone, web access, fax, mail or any other suitable means.

The system also provides a “duplicate information exchange” system that allows an individual to identify a third party to whom the individual's account/profile information may be sent or otherwise communicated. The individual's information may be sent to the third party when the initial account set-up and then sent again whenever the account information is updated. Such third parties may include, but are not limited to, caregivers, doctors, dentists, nurses, medical specialists, hospital administrative personnel, healthcare facilities, managed healthcare companies, pharmacists, pharmacies, healthcare insurers, healthcare networks, nursing homes, assisted living facilities, home healthcare organizations, call center or other value-add organizations. Communication of this information would be by email, phone, web access, fax or mail.

Although specific features of the invention are shown in some drawings and not others, this is for convenience only as some feature may be combined with any or all of the other features in accordance with the invention.

Other embodiments will occur to those skilled in the art and are within the following claims:

What is claimed is:

1. A system for ensuring that a person receives individual-specific health care and avoiding adverse drug reactions, comprising:
an information card on which critical need medical information, specific to said person, may be stored;
a means for storing said critical need medical information specific to said person on said card;
a means for reading said critical need medical information for the purpose of providing a health-related service to said person in view of said information read from said card; and
a means for updating said information stored on said card.

2. The system of claim 1, wherein said means for updating comprises a database, of at least some of said critical need medical information, that is electronically accessible by said person, and a card printer.

3. The system of claim 2 wherein said database is accessible by said person via a computer networked system.

4. The system of claim 1, wherein said means for updating is a database, comprising information about a plurality of people, that is accessible by said person by entering a personal identification code specific to said person, and wherein said person can only access said critical need medical information of said person.

5. The system of claim 4, wherein said means for updating allows said person to change said person's personal information code.

6. The system of claim 1, wherein said information card comprises an electronically-readable means for storing said medical information.

7. The system of claim 6, wherein said electronically-readable means comprises a magnetic strip.

8. The system of claim 6, wherein said electronically-readable means comprises a computer chip.

9. The system of claim 6, wherein said electronically-readable means comprises a memory means selected from a group consisting of a memory card and memory stick.

10. The system of claim 1, wherein said means for updating comprises a telephonically accessible database.

11. The system of claim 1, wherein said means for reading comprises a means for electronically reading said critical need medical information stored on said card.

12. The system of claim 1, wherein said means for updating is a stand-alone personal computer and a computer program residing in said computer that enable said person to update said person's medical information by entering updated information into said computer to update said information on said card.

13. The system of claim 12, further comprising a means for creating a new card with said updated information stored on said new card.

14. The system of claim 1, wherein said critical need medical information comprises at least one type of information specific to said person and selected from a group consisting of: allergies, prescriptions used by said person, non-prescription medications used by said person, devices implanted in said person, ailments of said person, and one or more electrocardiograms of said person.

15. The system of claim 1, wherein said card further comprises identification information specific to said person.

16. The system of claim 15, wherein said identification information comprises at least one type of information specific to said person and selected from a group consisting of: said person's primary care physician, personal emergency contact information, organ donation information, personal residential information, contact information for closest relative, health insurance information, and date critical need medical information was last updated.

17. A system for ensuring that a person receives individual-specific health care and avoiding adverse drug reactions, comprising:
an information card on which critical need medical information, specific to said person, may be stored;
a means for storing said critical need medical information specific to said person on said card; and
a means for enabling said person to electronically update said person's own medical information stored on said card.

18. The system of claim 17, wherein said means for storing comprises, an imprinting device for imprinting a plurality of raised letters and numbers on a surface of said card corresponding to at least some of said critical need medical information.

19. The system of claim 17, wherein said means for enabling said person to electronically update said person's own medical information stored on said card comprises a database electronically accessible by said person via a computer networked system, and further comprising a means for creating a new information card on which said person's updated medical information is stored.

20. The system of claim 19, further comprising a means for said person to communicate, via a computer networked system, with one or more value-added service structures capable of assisting said person to create, change and maintain said person's information.

21. The system of claim 20, wherein said service structures comprises one or more entities selected from a group consisting of: call-centers, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities, and caregivers.

22. The system of claim 17, further comprising a means for managing information exchange between said person and one or more service entities selected from a group consisting of: call-centers, doctors, emergency rooms, dentists, nurses, medical specialists, hospital administrative personnel, healthcare facilities, managed healthcare companies, pharmacists, pharmacies, healthcare insurers, healthcare networks, nursing homes, assisted living facilities, and caregivers.

23. The system of claim 17, wherein said means for enabling said person to electronically update said person's own medical information stored on said card comprises one or more kiosk stations.

24. The system of claim 23, further comprising an electronic database accessible via a computer networked system and wherein said kiosk stations comprise a means for accessing said database via said computer networked system.

25. The system of claim 17, further comprising a means for communicating at least a portion of said critical need medical information of said person to one or more third parties designated by said person.

26. The system of claim 25, wherein one or more of said designated third parties is selected from a group consisting of: family members, caregivers, doctor, dentists, nurses, medical specialists, hospital administrative personnel, healthcare facilities, managed healthcare companies, pharmacists, pharmacies, healthcare insurers, healthcare networks, nursing homes, assisted living facilities, home healthcare organizations, call center or other value-add organizations.

27. The system of claim 25, wherein said means for communicating is selected from a group consisting of: wired computer networked system, wireless computer networked system, satellite, cable, telephone, facsimile, and mail.

28. The system of claim 17, wherein said means for storing comprises a thermal printing device for printing at least some of said critical need medical information on a surface of said card.
